(19) United States
(12) Patent Application Publication
(10) Pub. No.: US 2007/0073219 A1
(43) Pub. Date: Mar. 29, 2007

(54) Retained needle structure

(76) Inventor: Chang-Ming Yang, Taiwan (CN)

Correspondence Address: HDSL, 4331 STEVENS BATTLE LANE, FAIRFAX, VA, 22033, US

(21) Appl. No.: 10/569,623

(22) PCT Filed: Aug. 28, 2003
(86) PCT No.: PCT/CN03/00727

§ 371 Date: Feb. 27, 2006

(51) Int. Cl. A61M 5/00

(52) U.S. Cl. 604/110

(57) ABSTRACT

A retained needle structure comprises a syringe needle having an inserting portion at a front end thereof, an injection needle inserted on the insertion portion, at least a fastening portion at a side rim thereof and a plug for reflowing at a rear end thereof; a catheter having a cannula, a flexible tube extending at a front end of the cannula and a stop portion disposed on an outer rim of a rear end of the cannula; a syringe cover having an aperture at a front end thereof, a plurality of gliding portions extending at an outer rim of a rear end thereof, the aperture having a plurality of hook portions disposed at a front end thereof and each of the gliding portions having a sliding slot formed thereon and a salient at a distal end thereof, wherein the catheter is sleeved on the inserting portion through the cannula thereof, the injection needle of the syringe needle is received within the flexible tube of the catheter and each of the fastening portions of the syringe needle is received within the respective sliding slots of the syringe cover so that the syringe cover slides on the syringe needle through the sliding slots.

### FIELD OF THE INVENTION

The present invention relates to a retained needle structure and particularly to a retained needle structure on which danger may be prevented when a nurse operates.

### DESCRIPTION OF THE PRIOR ART

A conventional retained needle structure comprises a syringe needle, a catheter and a syringe cover. The catheter is sleeved on the syringe needle and the syringe cover is disposed to cover a front end of the syringe needle and an outer rim of the catheter. When the retained needle structure is used, the syringe cover has to be moved first and then an injection needle on the syringe needle along with the catheter are catheterized into a specific portion of a patient body. Next, a flexible tube and a cannula of the catheter are pressed and then the syringe needle is pulled out. As such, the catheter is left on the injected portion of the patient and separated with the syringe needle. When the patient is to be actually injected with a liquid medicament, a duct is inserted into the cannula of the catheter and then the liquid medicament may be injected into the patient body. However, the blood of the patient will flood out from the catheter when the syringe needle and the catheter of the retained needle structure are separated with each other. In this regard, the injector has to insert the duct into the cannula of the retained needle structure very rapidly once the syringe needle is pulled in prevention of excess blood flooding from the injected portion of the patient. Unfortunately, this may lead to another problem that the injector is easy to be hurt by the injection needle of the syringe needle.

In view of the above discussion, the conventional retained needle structure still has some shortcomings to be addressed and thus has to be improved. Accordingly, the inventor of the present invention has paid an effort in the related field and finally developed successfully a retained needle structure of the present invention.

### SUMMARY OF THE INVENTION

It is, therefore, an object of the present invention to provide a retained needle structure on which danger may be prevented when a nurse operates.

It is another object of the present invention to provide a retained needle structure in which an injection needle associated therewith may be prevented from being inclined since a syringe needle thereof slides along a sliding slot to an inclination portion when the syringe needle and a catheter thereof are separated from each other, so that an injector associated therewith may be prevented from danger.

It is yet another object to provide a retained needle structure with which blood of a patient may be prevented from flooding inversely through the catheter when the syringe needle are separated with the catheter.

In accordance with the present invention, the retained needle structure comprises a syringe needle having an inserting portion at a front end thereof, an injection needle inserted on the insertion portion, at least a fastening portion at a side rim thereof and a plug for reflowing at a rear end thereof; a catheter having a cannula, a flexible tube extending at a front end of the cannula and a stop portion disposed on an outer rim of a rear end of the cannula; a syringe cover having an aperture at a front end thereof, a plurality of gliding portions extending at an outer rim of a rear end thereof, the aperture having a plurality of hook portions disposed at a front end thereof and each of the gliding portions having a sliding slot formed thereon and a salient at a distal end thereof, wherein the catheter is sleeved on the inserting portion through the cannula thereof, the injection needle of the syringe needle is received within the flexible tube of the catheter and each of the fastening portions of the syringe needle is received within the respective sliding slots of the syringe cover so that the syringe cover slides on the syringe needle through the sliding slots.

### BRIEF DESCRIPTION OF THE DRAWINGS

The drawings disclose several illustrative embodiments of the present invention which serve to exemplify the various advantages and objects hereof, and are as follows:

FIG. 1 is a first exploded diagram of a retained needle structure according to the present invention;

FIG. 1A is a second exploded diagram of the retained needle structure according to the present invention;

FIG. 1B is a third exploded diagram of the retained needle structure according to the present invention;

FIG. 2 is a schematic diagram of the retained needle structure according to the present invention;

FIG. 3 is a schematic diagram of the retained needle structure according to a first embodiment of the present invention;

FIG. 4 is a schematic diagram of the retained needle structure according to a second embodiment of the present invention;

FIG. 5 is a schematic diagram of the retained needle structure according to a third embodiment of the present invention;

FIG. 6 is a schematic diagram of the retained needle structure according to a fourth embodiment of the present invention;

FIG. 7 is a schematic diagram of the retained needle structure according to a fifth embodiment of the present invention;

FIG. 8 is a schematic diagram of the retained needle structure according to a sixth embodiment of the present invention;

FIG. 9 is a schematic diagram of the retained needle structure according to a seventh embodiment of the present invention;

FIG. 9A is a schematic diagram of the retained needle structure according to an eighth embodiment of the present invention;

FIG. 10 is a schematic diagram of the retained needle structure according to a ninth embodiment of the present invention;

FIG. 10A is a schematic diagram of the retained needle structure according to a tenth embodiment of the present invention;

FIG. 11 is an exploded diagram of another retained needle structure according to the present invention;

FIG. 12 is a schematic diagram of the another retained needle structure according to a first embodiment of the present invention;

FIG. 12A is a schematic diagram of the another retained needle structure according to a second embodiment of the present invention;

FIG. 12B is a schematic diagram of the another retained needle structure according to a third embodiment of the present invention;

FIG. 12C is a schematic diagram of the another retained needle structure according to a fourth embodiment of the present invention;

FIG. 13 is a schematic diagram of the another retained needle structure according to a fifth embodiment of the present invention;

FIG. 14 is a schematic diagram of the another retained needle structure according to a sixth embodiment of the present invention;

FIG. 15 is a schematic diagram of the retained needle structure according to a ninth embodiment of the present invention;

FIG. 16 is a schematic diagram of the retained needle structure according to an eleventh embodiment of the present invention;

FIG. 17 is a schematic diagram of the retained needle structure according to a twelfth embodiment of the present invention;

FIG. 18 is a schematic diagram of the another retained needle structure according to a seventh embodiment of the present invention;

FIG. 19 is a schematic diagram of the retained needle structure according to a thirteenth embodiment of the present invention;

FIG. 20 is a schematic diagram of a catheter of the retained needle structure according to a first embodiment of the present invention;

FIG. 21 is a schematic diagram of the catheter of the retained needle structure according to a second embodiment of the present invention; and

FIG. 22 is a schematic diagram of the catheter of the retained needle structure according to a third embodiment of the present invention.

### DETAILED DESCRIPTION OF THE PREFERRED EMBODIMENT

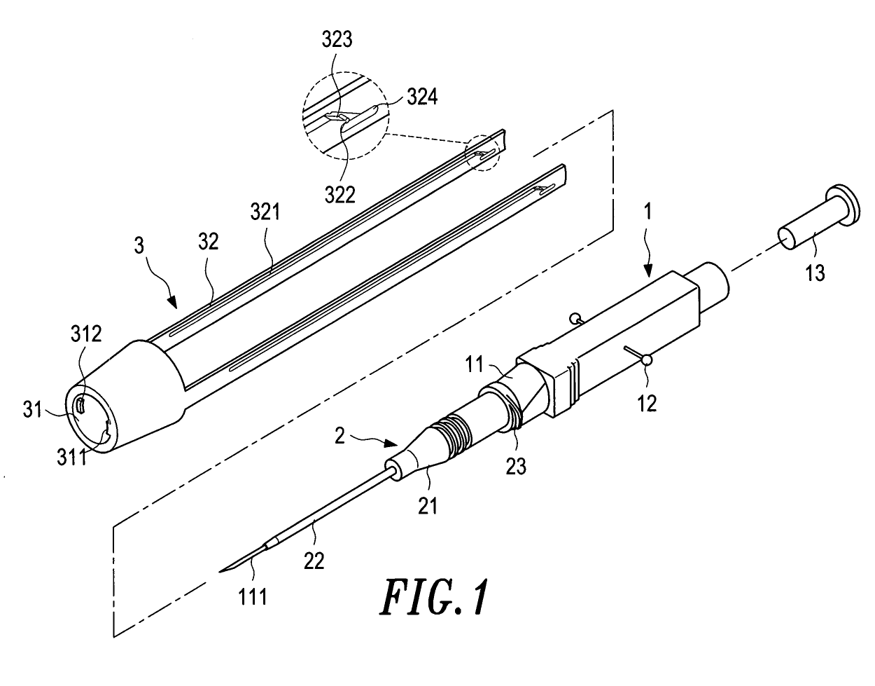

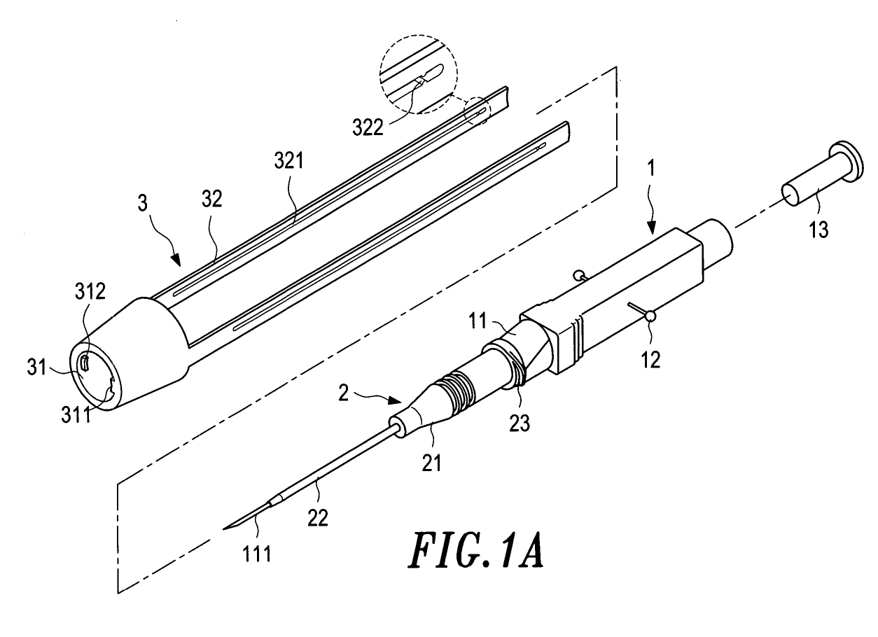

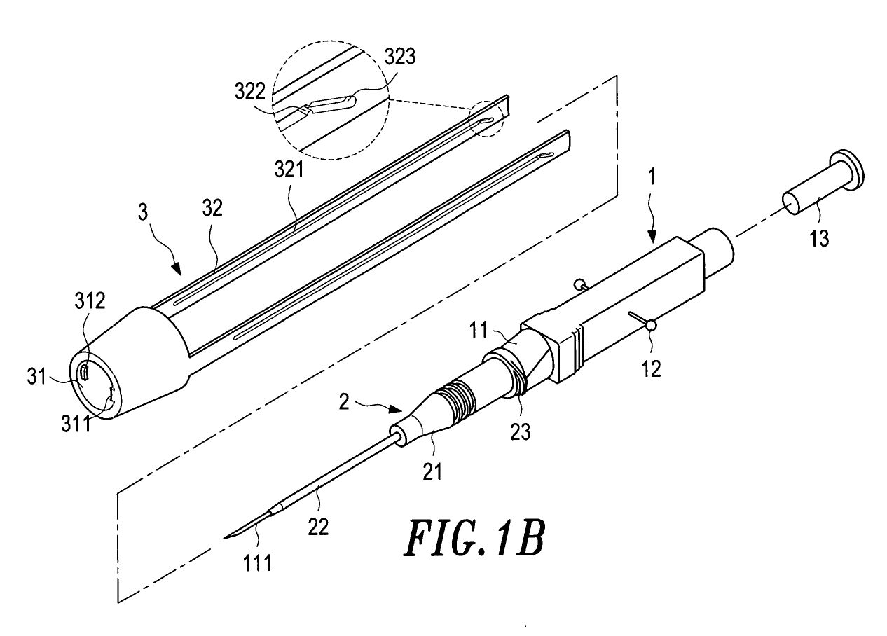

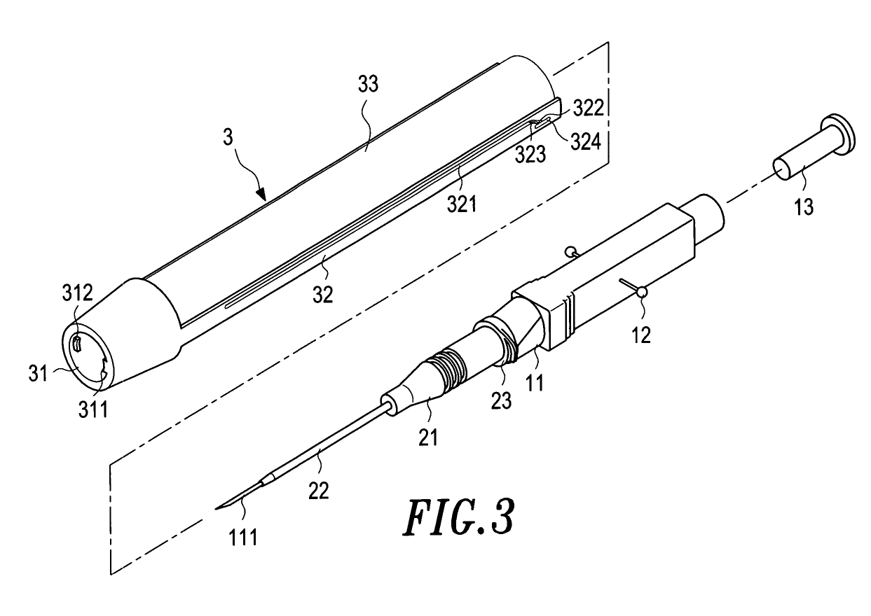

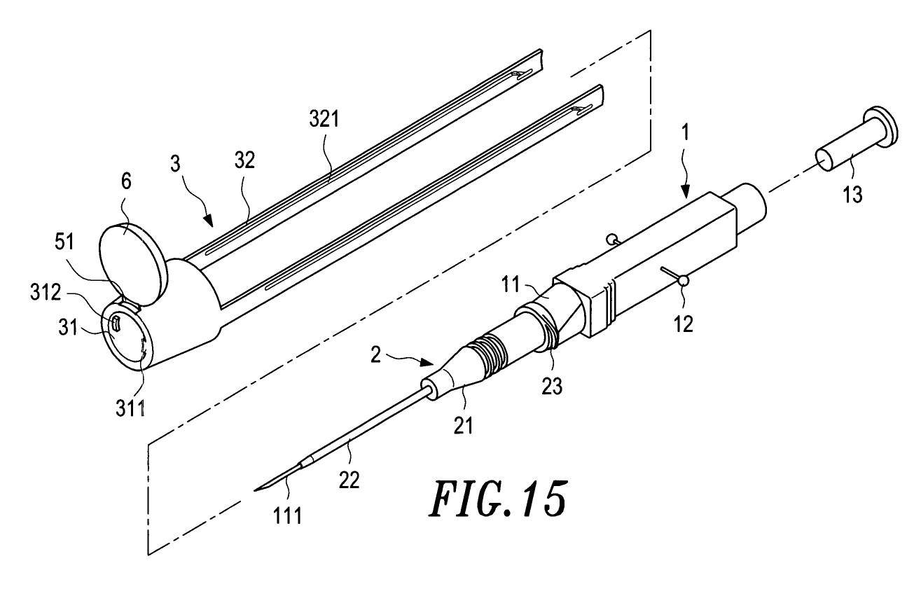

Referring to FIG. 1, FIG. 1A, FIG. 1B, FIG. 3 and FIG. 15, a retained needle structure of the present invention comprises a syringe needle 1, a catheter 2 and a syringe cover 3.

The syringe needle 1 has an inserting portion 11 at a front end thereof. On the inserting portion 11 is inserted an injection needle 111. On the syringe needle 1, one or more fastening portion 12 is disposed at a side rim thereof and a plug 13 for reflowing is inserted therein.

The catheter 2 has a cannula 21 having a flexible tube 22 extending from a front end thereof. A stop portion 23 is disposed at an outer rim of a rear end of the cannula 21. The catheter 2 is sleeved on the inserting portion 11 of the syringe needle 1. The injection needle 111 on the syringe needle 1 is received in the flexible tube 22 of the catheter 2.

The syringe cover 3 has an aperture 31 formed at a front end thereof. A plurality of hook portions 311, 312 are disposed at a front end of the syringe cover 3. A plurality of gliding portions 32 extend at an outer rim of a rear end of the syringe cover 3 corresponding to the fastening portion 12 of the syringe needle 1 in position and number. On each of the gliding portions 32, a sliding slot 321 is disposed. A salient 322 is disposed in the vicinity of a distal end of the sliding slot 321. When the fastening portion 12 of the syringe needle 1 slides along the sliding slot 321 of the syringe cover 3 to the distal end, the salient 322 serves to help seize the fastening portion 12. As such, the fastening portion 12 of the syringe needle 1 may be prevented from sliding out of the syringe cover 3 and hurting others. In addition, an outer sleeve 33 may be additionally disposed on an inner rim of each of the gliding portion 32 of the syringe cover 3 so as to cover an outer rim of the injection needle 111. A connection member 51 may be additionally disposed at the front end of the syringe cover 3 so that the syringe cover 3 may be connected with a lid 6.

In addition, a plurality of gliding portions 32 extend at an outer rim of the rear end of the syringe cover 3 corresponding to the fastening portion 12 of the syringe needle 1 in position and amount. A sliding slot 321 is formed on each of the gliding portions 32. A downward slant portion 323 is formed in the vicinity of the distal end of each of the gliding portions 32, shown in FIG. 1B. A salient 322 is formed on a front end of the downward slant portion 323. When the fastening portion 12 of the syringe needle 1 slides along the sliding slot 321 of the syringe cover 3 to the distal end of the syringe cover 3 and thus to the downward slant portion 323, the salient 322 serves to help seize the fastening portion 12 and the downward slant portion 323. As such, the injection needle 111 slants and thus the fastening portion 12 of the syringe needle 1 may be prevented from sliding out of the syringe cover 3 and hurting others.

In addition, a plurality of gliding portions 32 extend at the outer rim of the rear end of the syringe cover 3 corresponding to the fastening portion 12 of the syringe needle 1 in position and amount. A sliding slot 321 is formed on each of the gliding portions 32. A downward slant portion 323 is formed in the vicinity of a distal end of each of the gliding portions 32. A reception slot is formed on a rear end of the downward slant portion 323, shown in FIG. 1, and a salient 322 is formed on a front end of the downward slant portion 323. When the fastening portion 12 of the syringe needle 1 slides along the sliding slot 321 of the syringe cover 3 to the distal end of the syringe cover 3 and thus to the downward slant portion 323 and the reception slot 324, the salient 322 serves to help seize the fastening portion 12. As such, the fastening portion 12 of the syringe needle 1 may be prevented from sliding out of the syringe cover 3 and thus the injection needle 111 may be prevented from hurting others.

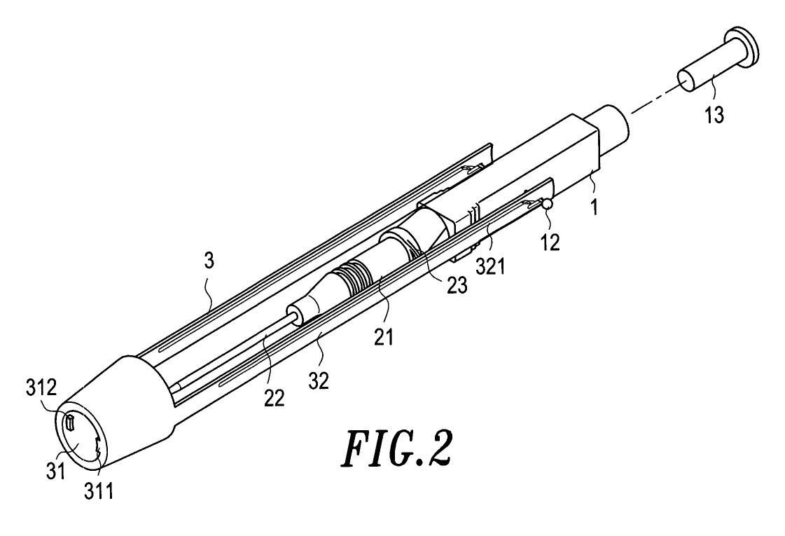

Referring to FIG. 2, when the syringe needle 1 and the syringe cover 3 combine, the fastening portion 12 of the syringe needle 1 slides along the sliding slot 321 of the syringe cover 3 to the distal end, the salient 322 serves to help seize the fastening portion 12. As such, the fastening portion 12 of the syringe needle 1 may be prevented from sliding out of the syringe cover 3 and thus the injection needle 111 may be prevented from hurting others. When the retained needle structure is used, the hook portions 311, 312 of the syringe cover 3 hook the stop portion 23 of the catheter 2 so that the catheter 2 is fixed in orientation.

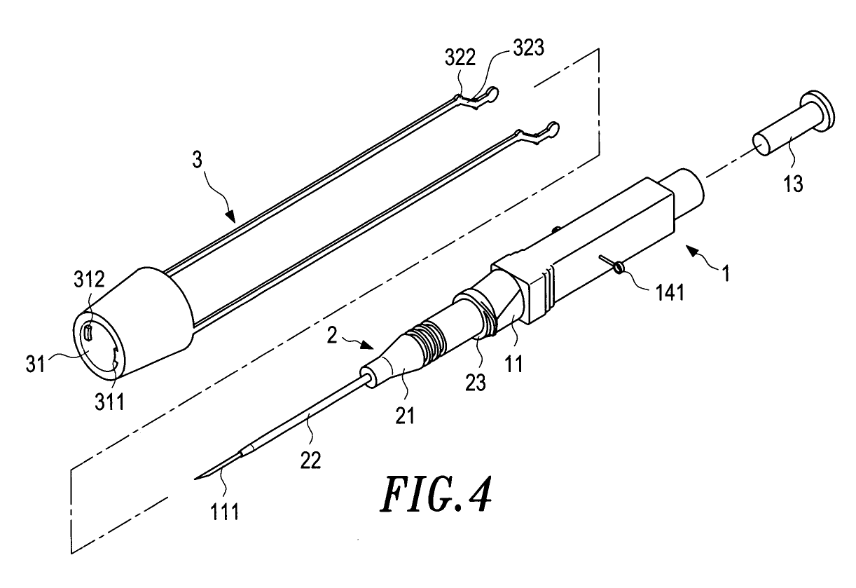

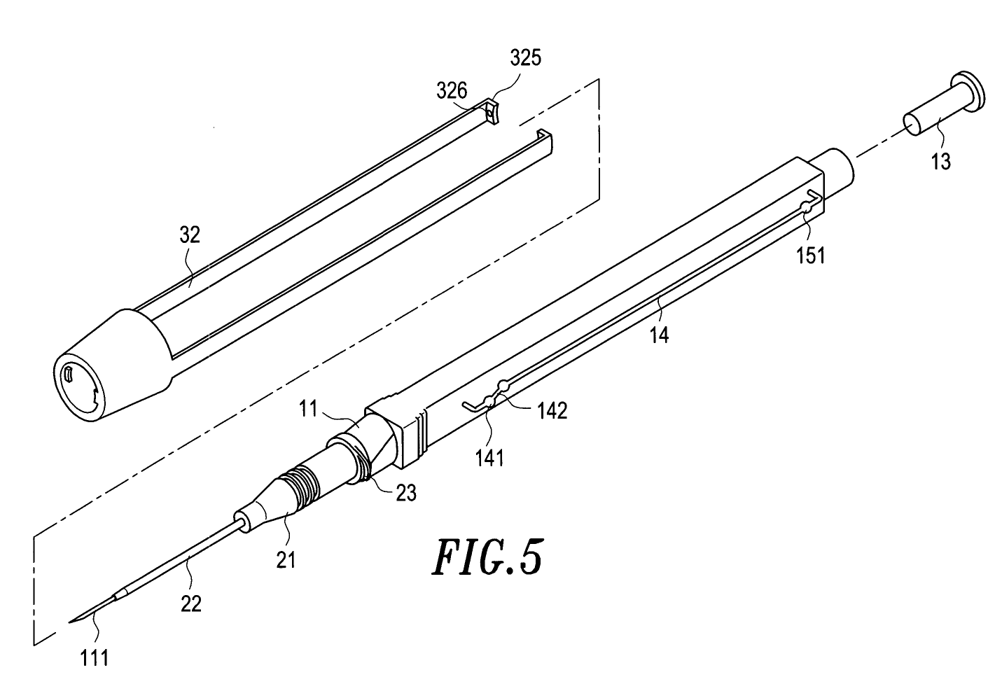

Referring to FIG. 4 and FIG. 5, in the retained needle structure of the present invention, fastening portions 12 on the syringe needle 1 is received in the respective sliding slots 321 on the syringe cover 3. When the retained needle structure is used, the syringe needle 1 slides on the sliding slot 321 of the syringe cover 3 by means of the fastening portion 12. Since the sliding slot 321 has a slant portion 323 and a salient 322 in the vicinity of the distal end thereof, only a slight force is provided will the fastening portion 12 pass through the salient 322. Since the fastening portion 12 of the syringe needle 1 slides on the sliding slot 321 of the syringe cover 3, the syringe cover 3 moves backwards to the rear side of the syringe needle 1, enabling the injection needle 111 and the catheter 2 to pass out from the aperture 31 of the syringe cover 3. Further, when the syringe cover 3 moves backwards to the rear side of the syringe needle 1, the hook portion 311 at the front end of the aperture 31 of the syringe cover 3 hooks the front rim of the syringe cover 3. At this time, the injection needle 111 and the flexible tube 22 of the catheter 2 are inserted together into a to-be-inject portion of the patient. Meanwhile, the cannula 21 and the flexible tube 22 of the catheter 2 are pressed and then the syringe needle 1 and the catheter 2 are separated and pulled out from the to-be-injected portion of the patient, causing the catheter 2 left at the to-be-injected portion of the patient. At this time, a duct is inserted into the cannula 21 of the catheter 2 and the liquid medicament is injected into the patient body by using the duct. Further, since the hook portions 311, 312 of the syringe cover 3 hook an outer rim of a front end of the stop portion 23, the syringe cover 3 slides forwards so that the injection needle 111 is covered in the syringe cover 3. At this time, a larger force is exerted to separate the syringe cover 3 and the catheter 2, and the fastening portion 12 of the syringe needle 1 is fixed by means of the slant portion 323 and the salient 322 at the distal end of the sliding slot 321. As such, the injection needle 111 is maintained at a covered state and thus the nurse involved in the injection task will not be hurt by the catheter 2.

In addition, the fastening portion 12 of the syringe needle 1 may be a ring shape 121 and the sliding portion 32 of syringe cover 3 may be a sliding tube 324, which may be combined together to form a sliding structure. The fastening portion 32 of the syringe needle 1 may be an “n”-shaped guide rail 14 and has a slant portion 142 at the front end thereof. A salient 141 is formed on the slant portion 142 and a bump 325 having a hole is formed on the distal end of the sliding portion 32 of the syringe cover 3, which are combined to form a sliding structure.

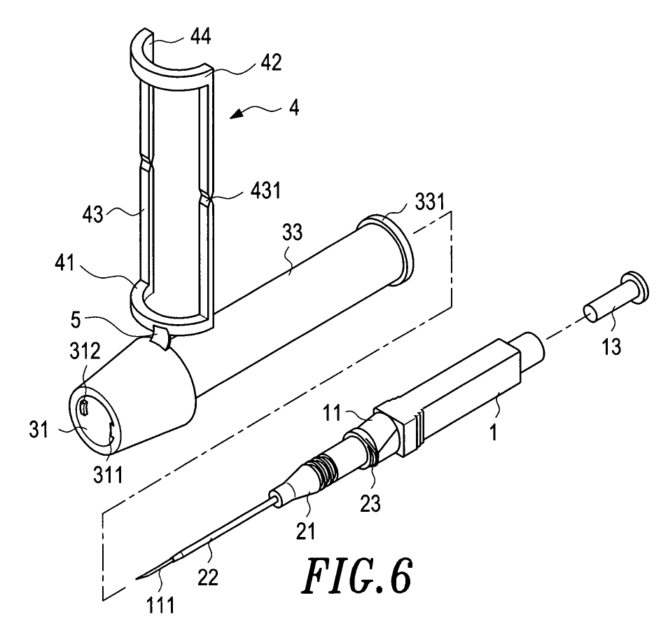

Referring to FIG. 6, the syringe cover 3 has a stop structure 4 formed on the front end thereof. The stop structure 4 is composed of a front stop body 41, a rear stop body 42 and one or more curved piece 43, wherein the curved piece 43 is connected between the front stop body 41 and the rear stop body 42 and a curved portion 431 is formed at a central portion of the curved piece 43. The front stop body 41 has a front end combined with the syringe cover 3 through a connection member 5. The rear stop body 42 has a rear end combined with a fastening seat 331 of the syringe cover 3.

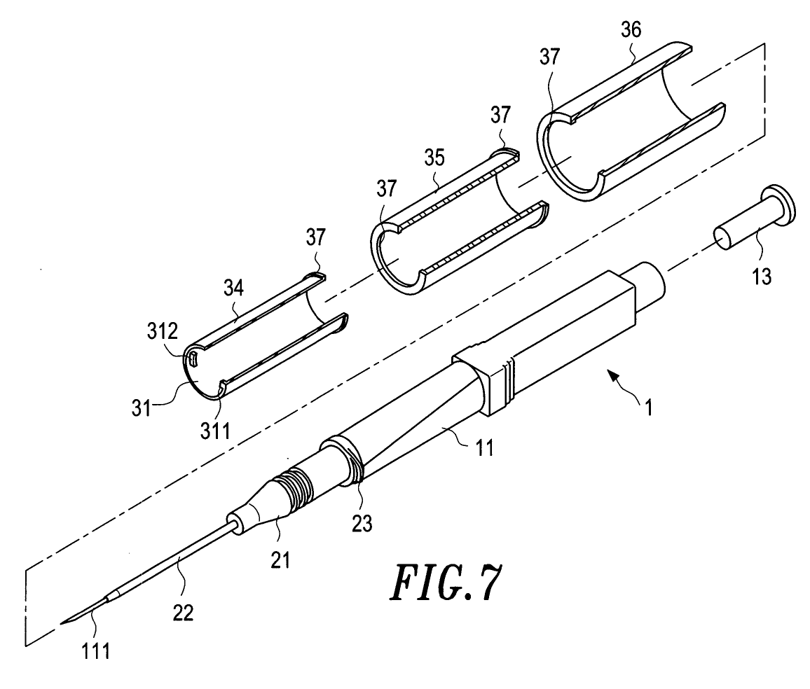

Referring to FIG. 7, the syringe needle 1 may do without the fastening portion 12 and the syringe cover 3 may be replaced with an inner rim sleeve 34, one or more intermediate sleeve 35 and an outer rim sleeve 36. The intermediate sleeve 35 is sleeved on the inner rim sleeve 34 and the outer rim sleeve 36 is sleeved on the intermediate sleeve 35. The outer sleeve 36 has a rear end sleeved on the outer rim of the syringe needle 1. A ring-shaped protrusion 37 is formed on an outer rim at a rear end of the inner rim sleeve 34 and the intermediate sleeve 35. Further, a plurality of hook portions 311, 312 are disposed on a front end of the inner rim of the inner rim sleeve 34.

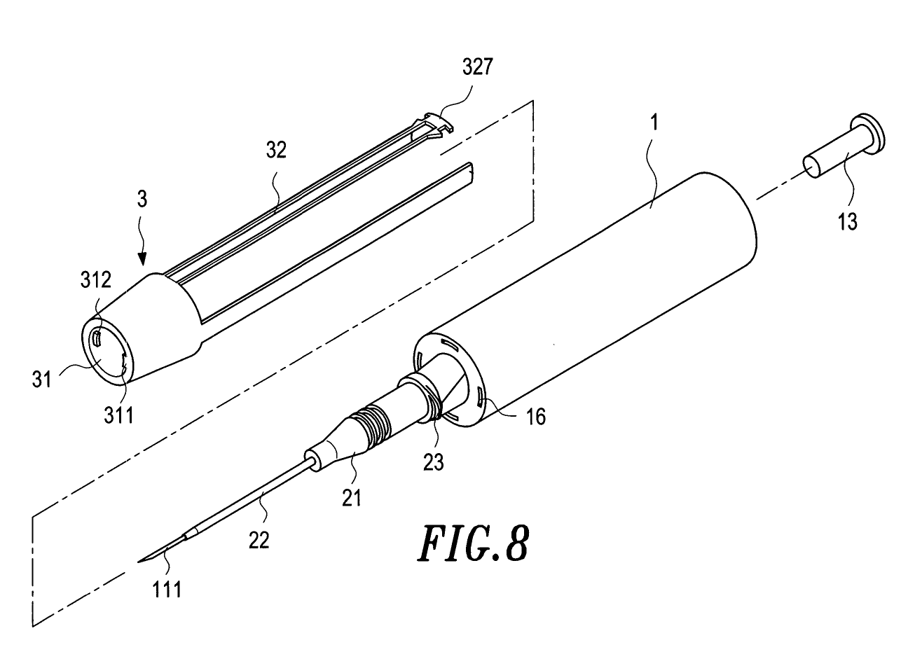

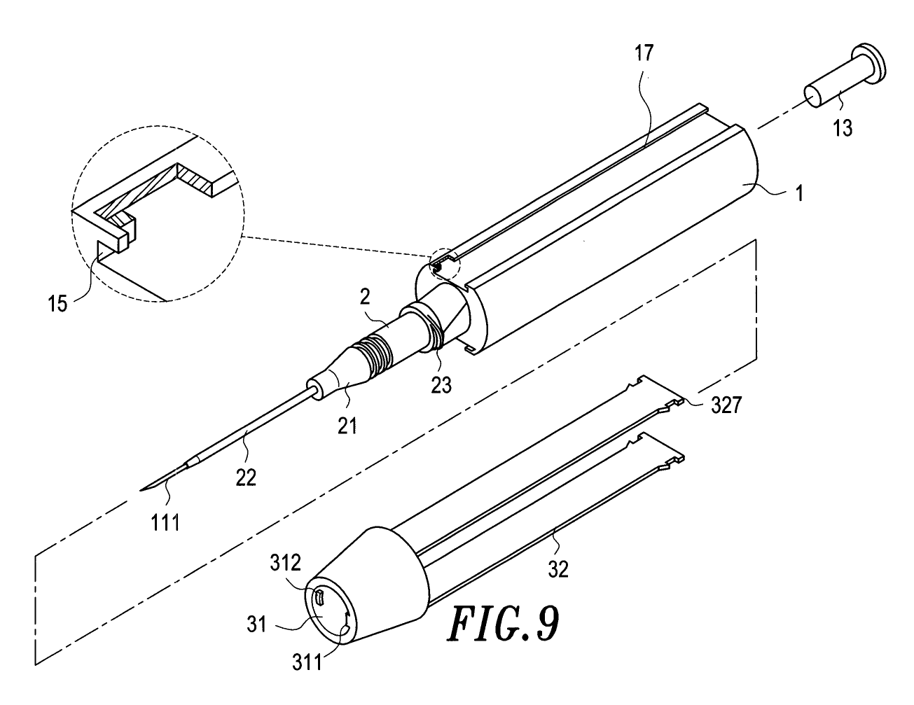

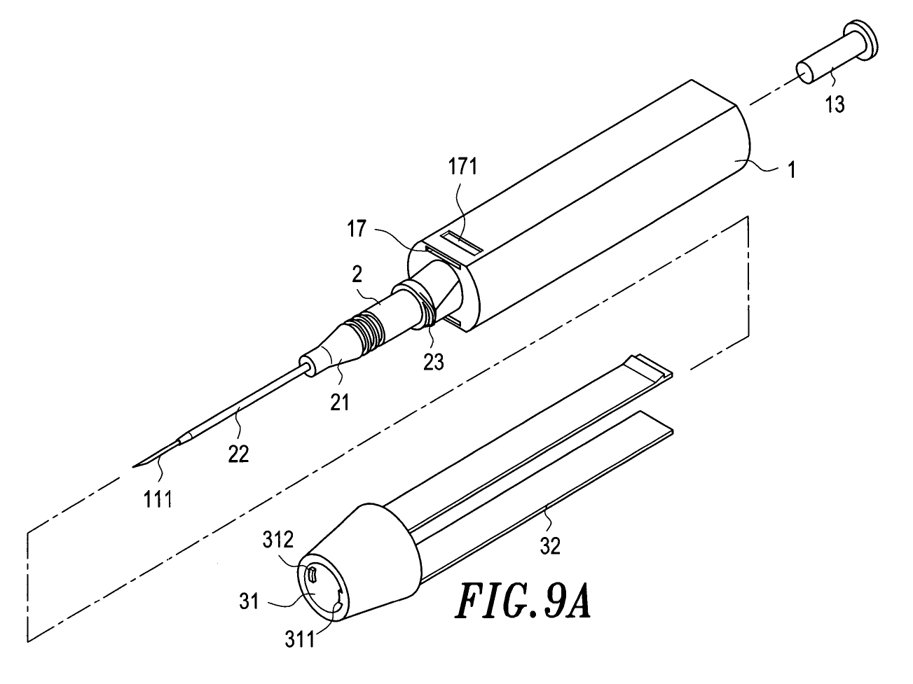

Referring to FIG. 8 and FIG. 9, the syringe needle 1 may do without the fastening portion 12 but have a plurality of guide slots 16 formed therein, wherein each of the guide slots 16 is allowed to be inserted with the respective gliding portion 32 of the syringe cover 3. Further, a plurality of stop bodies 327 are disposed on the rear end of either or all of the gliding portions 32, wherein the gliding portion 32 may be a hollow portion so that the sliding portion 32 may be inserted into the guide slots 16 of the syringe needle 1. In addition, a plurality of slide rails 17 may be disposed on the outer rim of the syringe needle 1. The gliding portion 32 at the rear end of the syringe cover 3 may be inserted into each of the slide rails 17. A plurality of stop bodies 327 are formed on the rear end of either or all of the gliding portions 32. At the front end of the slide rail 17, a stop body 15 is formed.

Referring to FIG. 9, a plurality of slide slots 17 are formed in the syringe needle 1, each of which may be inserted with the gliding portion 32 of the syringe cover 3. At a distal end of the gliding portion 32, a bump is formed. A fastening slot 171 is provided on an upper end of the syringe cover 3. When the gliding portion 32 of the syringe cover 3 is sleeved, the bump of the gliding portion 32 slides into the fastening slot 171 of the syringe cover 3. As such, the syringe cover 3 may be fixed and the injection needle 111 may be prevented from hurting others.

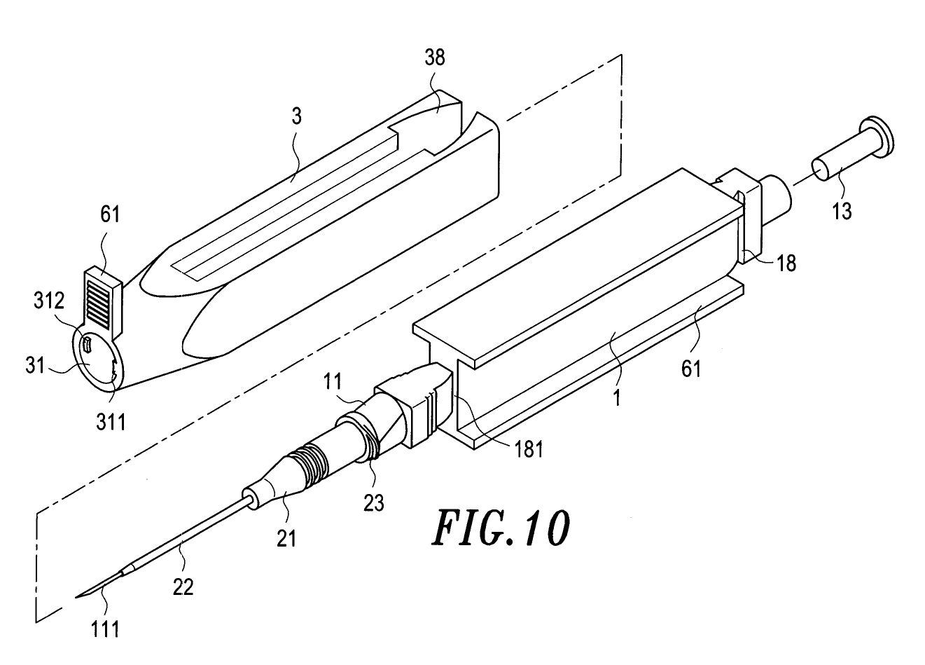

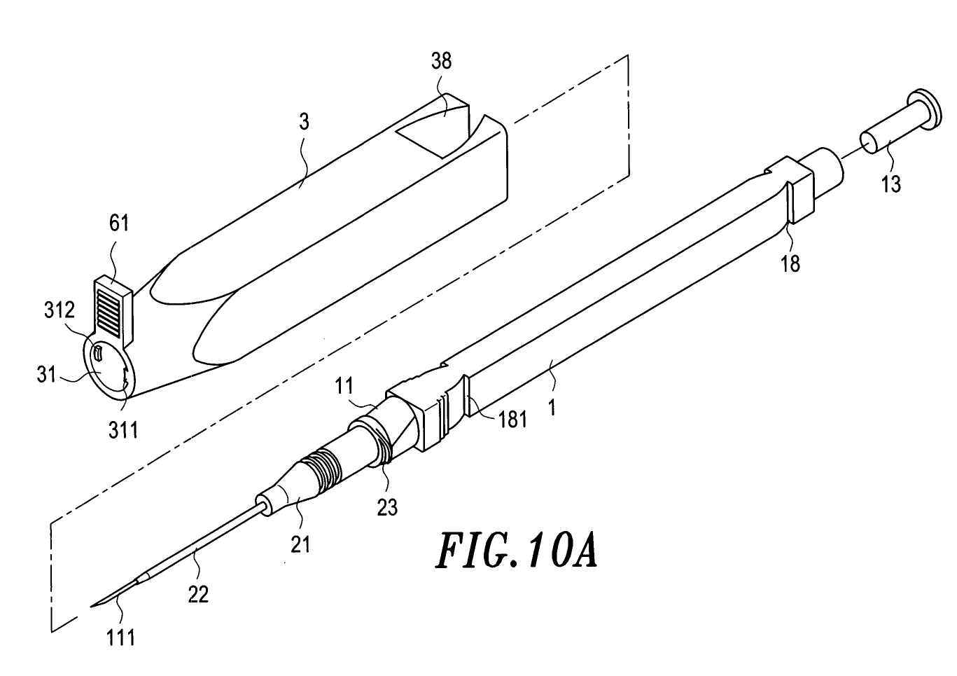

Referring to FIG. 10 and FIG. 10A, the syringe needle 1 may do without the fastening portion 12, which may be replaced with a stop portion 181 disposed at the front end of the outer rim of the syringe needle 1, as opposed to a reception portion 18 disposed at the rear end of the outer rim of the syringe needle 1. On a respective upper end of the syringe cover 3 and the syringe needle 1, an urging piece 61 is additionally provided. In addition, the syringe cover 3 is hollowed at a central portion thereof and disposed with a fastening hook 38 at the rear end for hooking with the reception portion 18 and the stop portion 181. Thus, when the urging piece 61 slides on the front or rear end of the syringe needle 1, the syringe cover 3 may be fixed at the syringe needle 1. Further, after the retained needle structure is used, the syringe cover 3 is pushed to the front end of the syringe needle 1 by the urging piece 61(or the syringe needle 1 is pulled backwards by the urging piece 61) so that the fastening hook 38 hooks the stop portion 181. At this time, the stop portion 181 urges behind the fastening hook 38 at a rear wall thereof and stops the syringe cover 3 from moving backwards. As such, the syringe cover 3 covers on the outer rim of the injection needle 111 and thus prevents the injection from being exposed and hurting others.

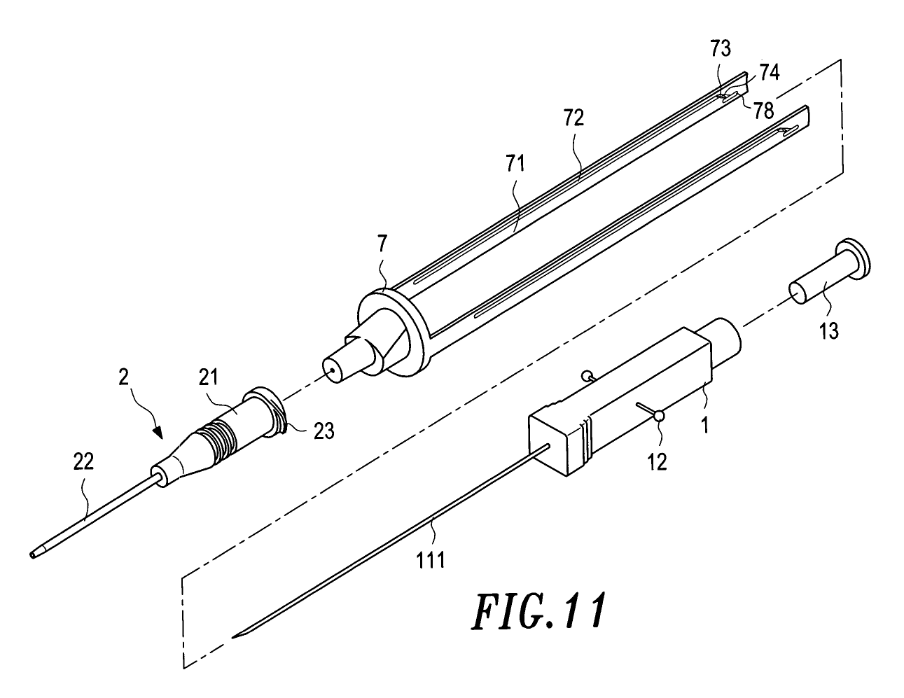

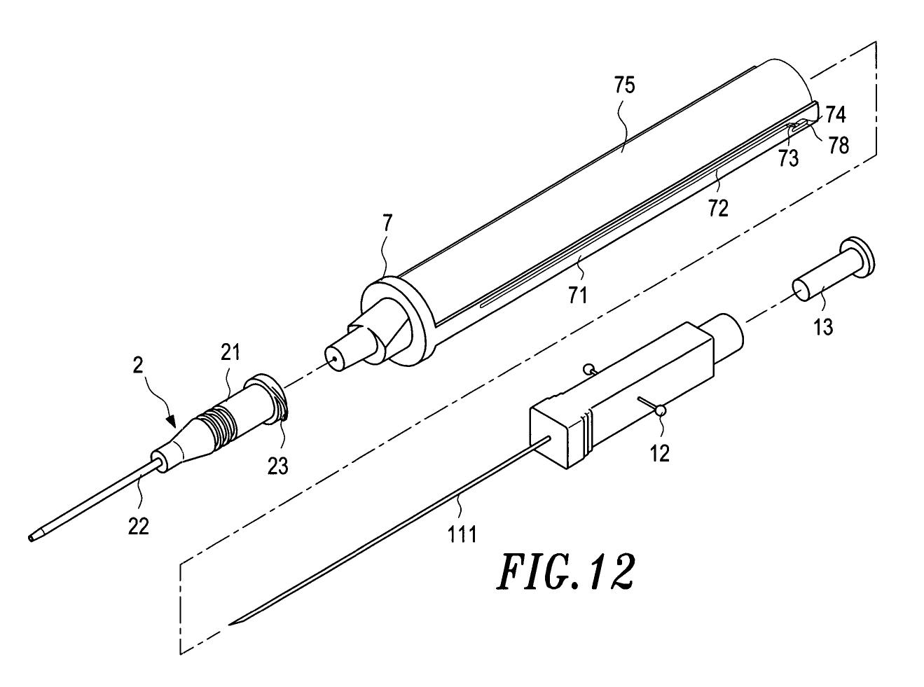

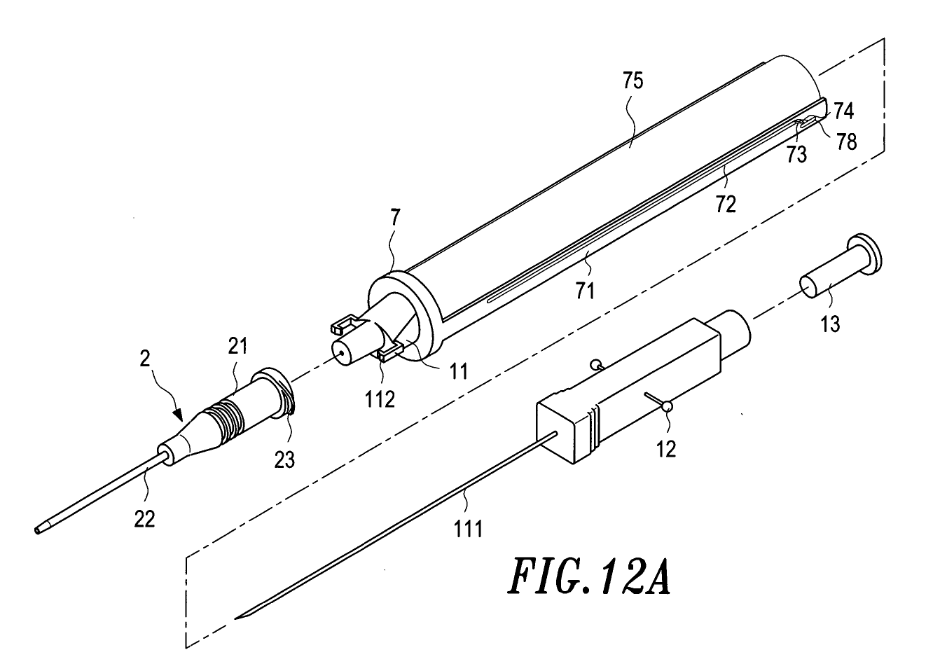

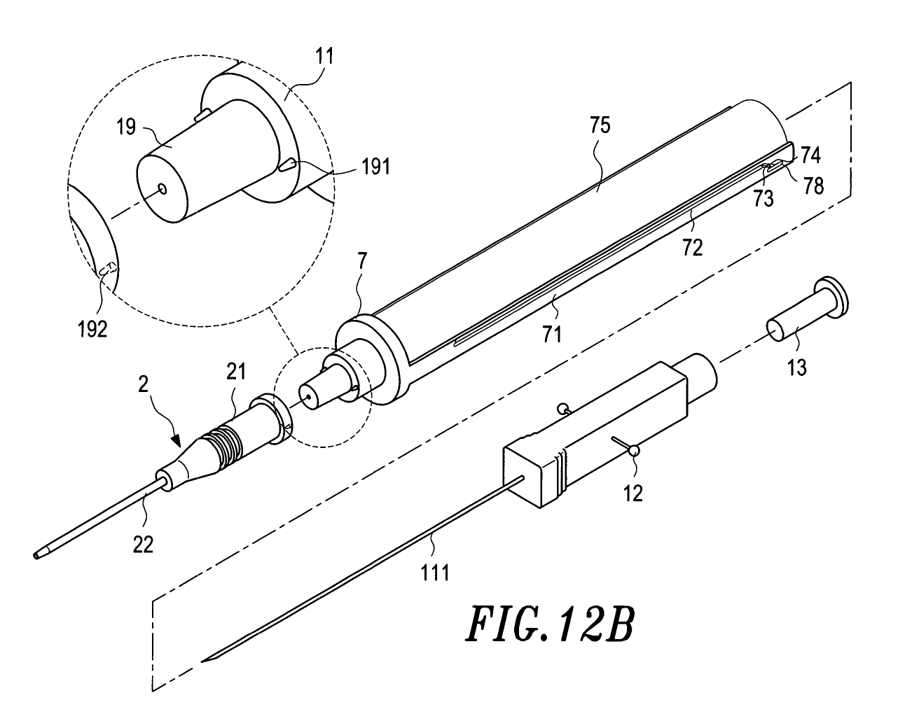

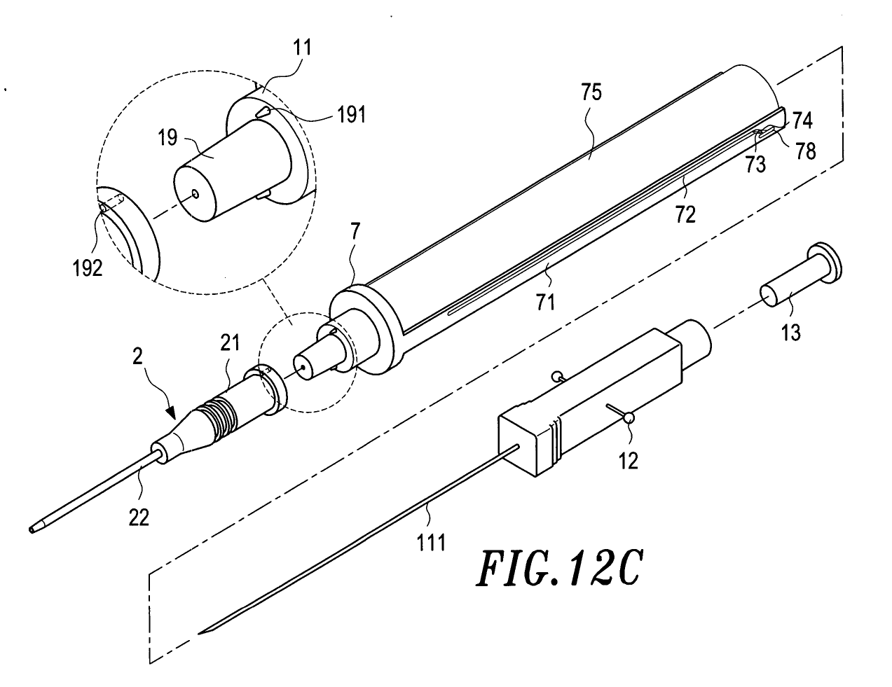

Referring to FIG. 11 and FIG. 12 through FIG. 12C, the retained needle structure comprises a syringe needle 1, a catheter 2 and a syringe cover 7. The syringe needle 1 has an injection needle 111 disposed at the front end thereof, one or more fastening portion 12 disposed at a side rim thereof and a plug 13 for reflowing inserted at the rear end thereof.

The catheter needle 2 has a flexible tube 22 extending at the front end of a cannula 21. A top portion 23 is disposed on an outer rim at the rear end of the cannula 21.

The syringe cover 7 has a plurality of gliding portions 32 extending at the outer rim of the rear end of the syringe cover 7 corresponding to the fastening portion 12 of the syringe needle 1 in position and amount. A sliding slot 72 is formed on each of the gliding portions 32. A salient 74 is formed near the distal end of the sliding slot 72. When the fastening portion 12 of the syringe needle 1 slides along the sliding slot 72 of the syringe cover 7 to the distal end of the syringe cover 7, the salient 374 serves to help seize the fastening portion 12. As such, the fastening portion 12 of the syringe needle 1 may be prevented from sliding out of the syringe cover 3 and thus the injection needle 111 may be prevented from hurting others. A downward slant portion 73 is formed at the distal end of sliding slot 72. A salient 74 is formed on the front end of the downward slant portion 73. When the fastening portion 12 of the syringe needle 1 slides along the sliding slot 72 of the syringe cover 3 to the downward slant portion 73, the downward slant portion 73 makes the injection needle 111 slants at the syringe cover 7 so that the injection needle 111 of the syringe needle 1 may be prevented from hurting others. Or, the downward slant portion 73 is formed on the distal end of the sliding slot 72 and a reception slot 78 is formed on the downward slant portion 73. Further, a salient 74 is formed on the distal end of the downward slant portion 73. When the fastening portion 12 of the syringe needle 1 slides along the sliding slot 72 of the syringe cover 7 to the reception slot 78 of the downward slant portion 73, the salient 74 help seize the fastening portion 12. As such, the fastening portion 12 of the syringe needle 1 may be prevented from sliding out of the syringe cover 7 and thus the injection needle 111 may be prevented from hurting others.

The syringe cover 7 is sleeved on the front end of the syringe needle 1 and the injection needle of the syringe needle 1 runs through the syringe cover 7. The catheter 2 is sleeved on the syringe cover 7 through the cannula 21. All the fastening portions of the syringe needle 1 are received in the respective sliding slots 72. By means of the sliding slot 72, the syringe cover 7 slides on the syringe cover 7. In addition, an outer sleeve 75 may be disposed on the outer rim of the injection needle 111 and within the inner rim of each of the gliding portions 71 of the syringe cover 7. One or more fastening piece 112 is disposed on the inserting portion 11 of the syringe cover 7. The fastening piece 112 and the stop portion 23 of the catheter 2 are fastened to each other so that the catheter 2 and the syringe cover 7 are fixed to each other. In addition, one or more salient 191 or indentation 192 may be disposed on the injection portion 1 of the syringe cover 7 to mate with one or more indentation 192 or salient 191 so that the catheter 2 and the syringe cover 7 may be more firmly fixed together. Further, the indentation 192 may also runs directly through the insertion portion 11 or the stop portion 23.

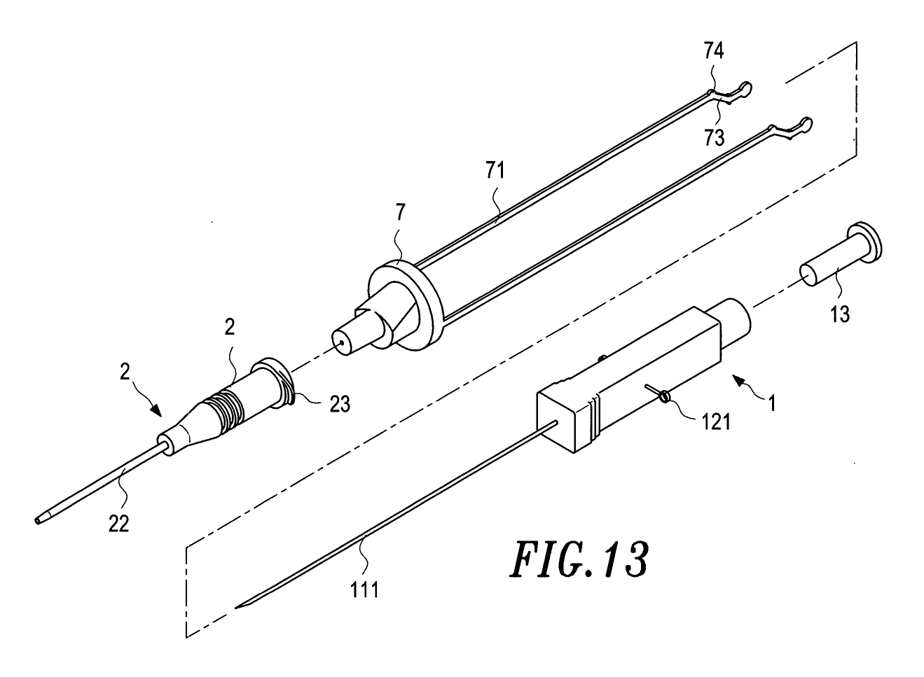

Referring to FIG. 13, the fastening portion 12 on the syringe needle 1 may be replaced with a guide structure 121 with a circular hole. The gliding portion 71 at the rear end of the syringe cover 7 is inserted roundly within the circular hole of the guide structure 121. At the distal end of each of the gliding portion 71, a slant portion 73 is formed. A salient 74 is formed at the front end of the slant portion 73.

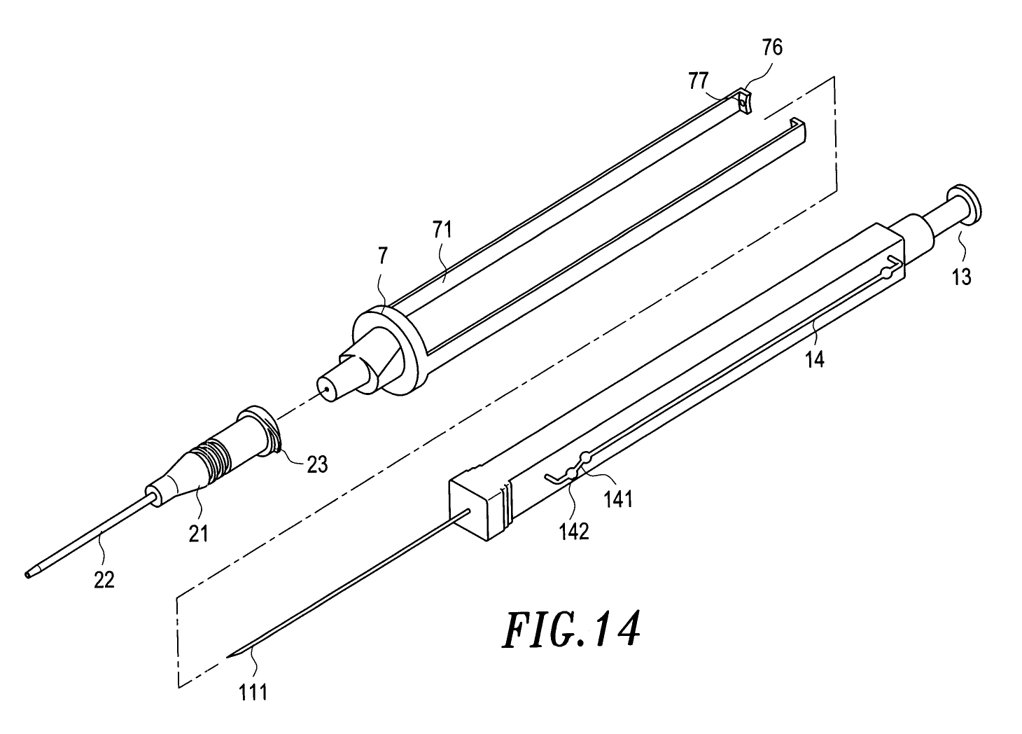

Referring to FIG. 14, the syringe needle 1 may do without the fastening portion 12 but have a plurality of “n”-shaped guide rail 14 extending at a side thereof and a salient 141 at a position near the front end of the “n”-shaped guide rail 14. In addition, a salient 76 having a hole 77 is formed at the rear end of each of the gliding portions 71 located at the rear side of the syringe cover 7, wherein each of the holes 77 is inserted within each of the “n”-shaped guide rail 14.

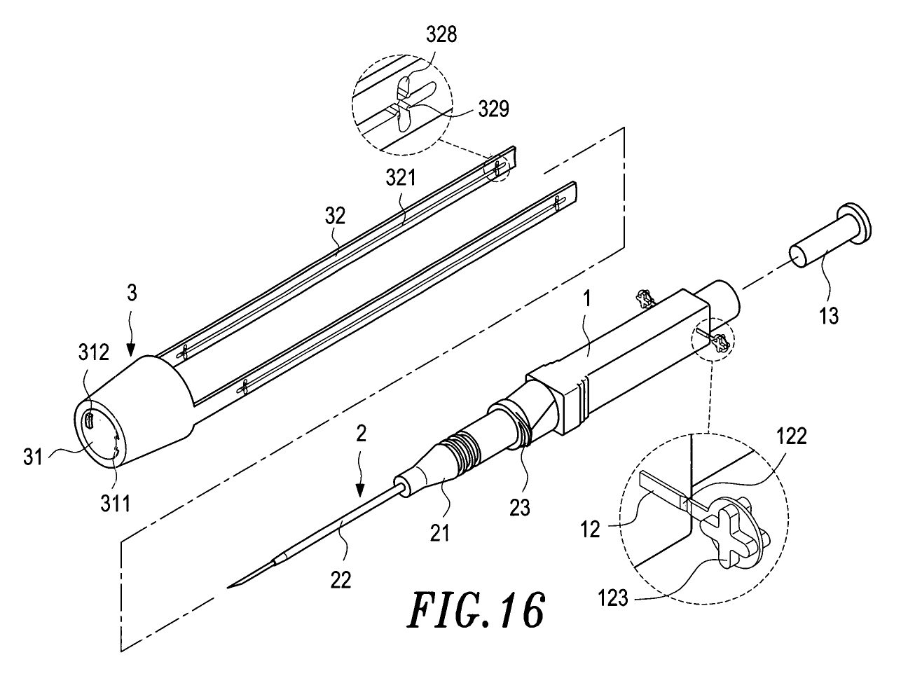

Referring to FIG. 16, a plurality of fastening holes 328 are formed at the sliding slot 321 of the gliding portion 32 at the rear end of the catheter 3. A protruding circular structure 329 is formed at an joint of the sliding slot 321 and each of the fastening holes 12. The fastening portion 12 of the syringe needle 1 has a curved portion 122. On a top end of the curved portion 122, a fastening piece 123 is disposed, which may be fastened to within the fastening hole 328. Before the retained needle structure is used, the curve portion 122 is inserted at a position where a rear end of the fastening hole 328 runs through the syringe cover 3, and moved within the sliding slot 321 so that the syringe cover 3 is sleeved on the outer rim of the syringe needle 1. Then, the fastening piece 123 at an end of the curved portion 122 is fastened into the fastening hole 328 located at the rear end of the sliding slot 321. Further, the protruding circular structure 329 at a joint of the sliding slot 321 and the fastening hole 328 urges against the fastening piece 123 so that the fastening piece 123 urges and is thus fixed within the fastening hole 328. As such, the injection needle 111 is covered with the syringe cover 3. Only by separating the fastening pieces 123 and the fastening hole 328 at the rear end of the sliding slot 321 can the retained needle structure be used.

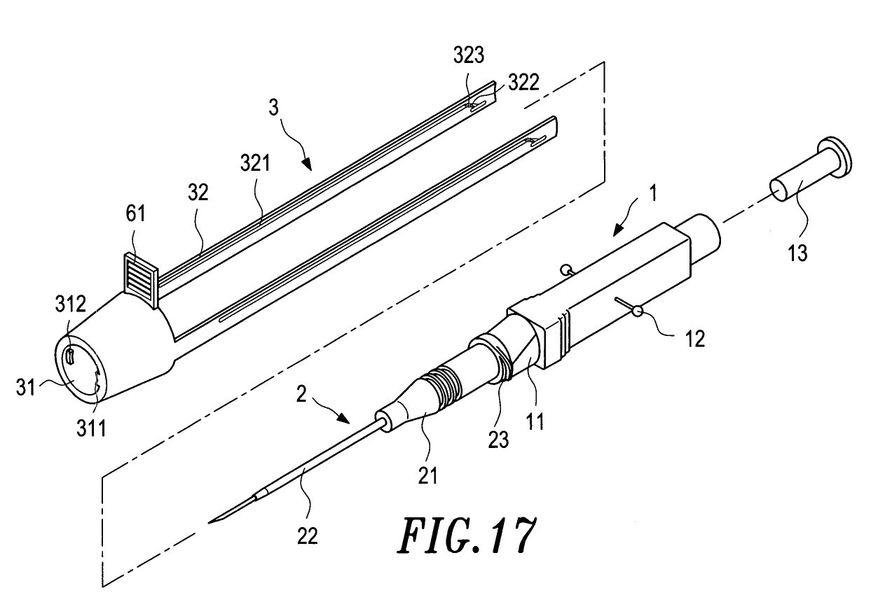

Referring to FIG. 17, the fastening portions 12 on the syringe needle 1 is received within the respective sliding slots 321 on the syringe cover 3. When the retained needle structure is used, the syringe needle 1 slides within the sliding slot 321 of the syringe cover 3 by means of the fastening portions 12. Since a slant portion 323, a reception slot 324 and a salient 322 are formed at a position near the distal end of the sliding slot 321, the fastening portion 12 has to be subject to a slight force so that it may pass through the salient 322.

Since the syringe needle 1 slides within the sliding slot 321 of the syringe cover 3 by means of the fastening piece 12, the syringe cover 3 moves backwards to behind the syringe needle 1 and the injection needle 111 and the catheter 2 passes through the aperture 31 of the syringe cover 3. When the syringe cover 3 moves backwards to behind the syringe needle 1, the aperture 31 of the syringe cover 3, a hook portion 311 at the front end of the aperture 31 of the syringe cover 3 has to be hooked at the front end of the stop portion 23 of the catheter 2. At this time, the injection needle 111 and the flexible tube 22 of the catheter 2 are inserted together into the to-be-injection portion of the patient. When the injection needle 111 is to be pulled out and the syringe needle 1 and the catheter 2 are to be separated from each other, the syringe needle 1 has to be moved along the sliding rail 321 of the syringe cover 3 by means of the urging piece 61. At this time, the syringe cover 3 slides forwards so that the injection needle 111 is covered within the syringe needle 3. At this time, a larger force is required to separate the syringe cover 3 from the catheter 2. Meanwhile, the fastening portion 12 of the syringe needle 1 is pushed to within the reception slot 324 by means of the salient 322 and the slant portion 323 at the distal end of the sliding slot 321.

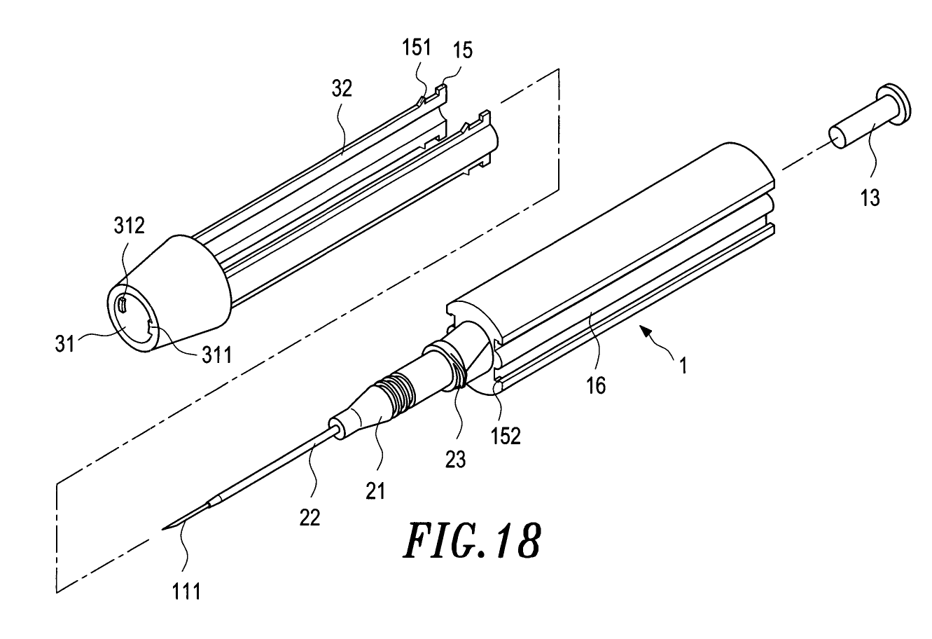

Referring to FIG. 18, the syringe needle may do without the syringe needle 1 but have a guide slot 16 formed in the syringe needle 1 where the guide slot 16 and the gliding portion 32 of the syringe cover 3 mate with each other in topology. At a front end of the guide slot 16, a stop body 15 is disposed, which is fastened with the stop body 15 located at the rear end of the gliding portion 32 of the syringe cover 3. As such, the syringe needle 1 and the syringe cover 3 may be prevented from separating from each other.

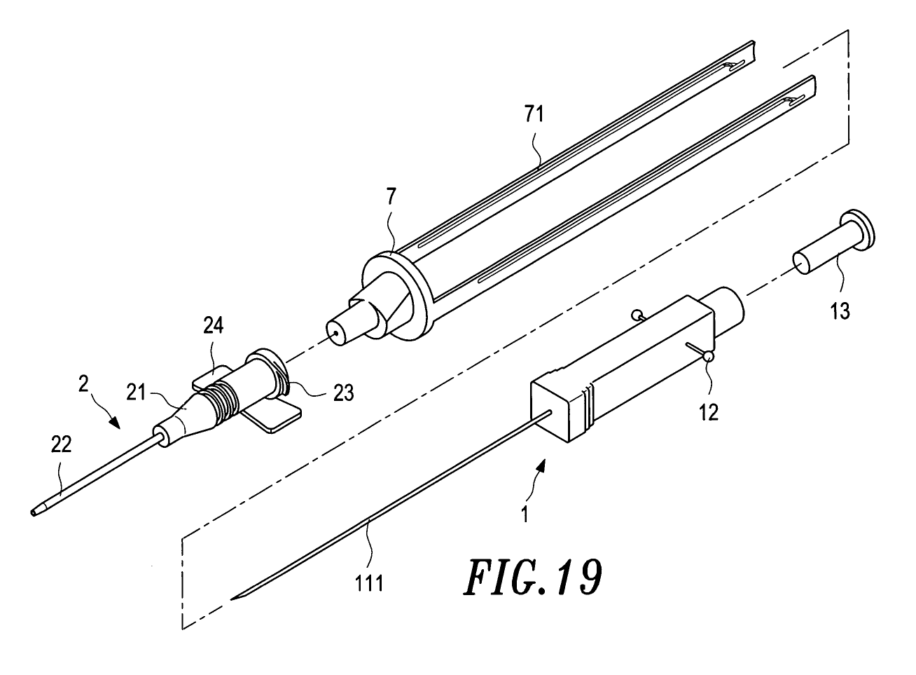

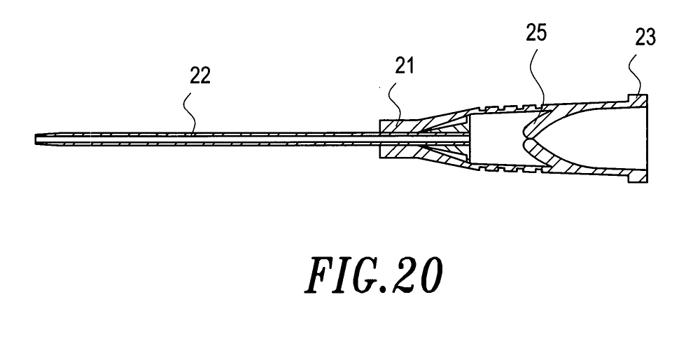

Referring to FIG. 19 through 20, the catheter 2 may additionally have a tongue-like piece 25 therein so that the injection needle 111 may be pulled out to receive different injection means. In addition, an adhesive piece 24 being adhesive may be stuck on two sides of the catheter 2 so that the catheter 2 can be stuck on the injected portion of the patient.

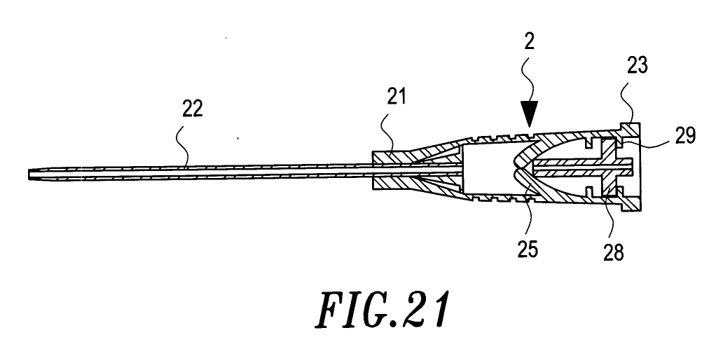

Referring to FIG. 21, the catheter 2 has a fixation piece 25 having an urging piece 28 at a front end thereof. The urging piece 28 has a hollow tube and extends between the fixation piece 25 and the stop body 29. When an object externally connected urges against the urging piece 28, the fixation piece 25 is opened by the urging piece 28 to facilitate the injected fluid flow passing therethrough.

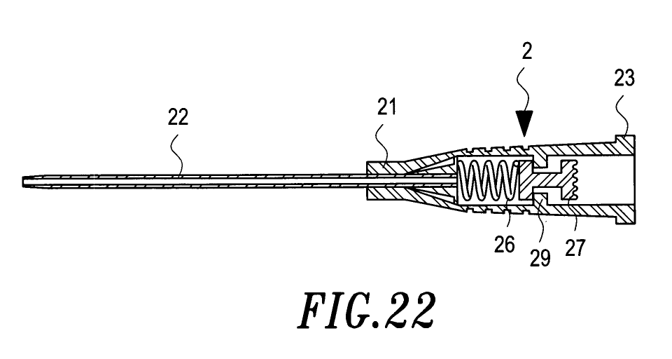

Referring to FIG. 22, the catheter 2 has a spring 26 therein, which has an urging piece 27 formed at a front end thereof. The urging piece 27 has an irregular surface in topology and disposed between the spring 26 and the stop body 29. When an object connected externally urges the urging piece 27 and thus presses the spring 27, the urging piece 27 leaves from the stop body 29 and thus a gap is formed, through which the injection fluid flow passes.

As compared to the prior art, the retained needle structure provides the following advantages: 1. Since the injection needle is covered by the syringe cover when the syringe needle is being separated, the nurse involved may be prevented from being hurt. 2. Since the inverse flow stopping structure or piston is formed within the retained needle structure, the blood of the patient can be prevented from flowing inversely when the syringe needle is being separated from the retained needle structure.

Many changes and modifications in the above described embodiment of the invention can, of course, be carried out without departing from the scope thereof. Accordingly, to promote the progress in science and the useful arts, the invention is disclosed and intended to be limited only by the scope of the appended claims.

I claim:

1. A retained needle structure, comprising:
a syringe needle having an inserting portion at a front end thereof, an injection needle inserted on the insertion portion, at least a fastening portion at a side rim thereof and a plug for reflowing at a rear end thereof;
a catheter having a cannula, a flexible tube extending at a front end of the cannula and a stop portion disposed on an outer rim of a rear end of the cannula;
a syringe cover having an aperture at a front end thereof, a plurality of gliding portions extending at an outer rim of a rear end thereof, the aperture having a plurality of hook portions disposed at a front end thereof and each of the gliding portions having a sliding slot formed thereon and a salient at a distal end thereof,
wherein the catheter is sleeved on the inserting portion through the cannula thereof, the injection needle of the syringe needle is received within the flexible tube of the catheter and each of the fastening portions of the syringe needle is received within the respective sliding slots of the syringe cover so that the syringe cover slides on the syringe needle through the sliding slots.

2. The retained needle structure as claimed in claim 1, wherein the front end of the syringe cover has a connection member connected with a lid.

3. The retained needle structure as claimed in claim 1, wherein each of the gliding potions of the syringe cover has a sliding slot thereon and a downward slant portion formed at a position near the distal end thereof.

4. The retained needle structure as claimed in claim 4, wherein the downward slant potion has a reception slot at a lower end thereof.

5. The retained needle structure as claimed in claim 1, wherein each of the fastening portions of the syringe needle is a ring shaped structure and each of the gliding portions is a stripe-shaped sliding tube combined with the respective one of the fastening portions as a gliding structure.

6. The retained needle structure as claimed in claim 1, wherein each of the fastening portions of the syringe needle is an “n”-shaped guide rail having a slant portion at a front end thereof, the slant portion having a salient formed thereon and each of the gliding portion of the syringe cover having a bump, the bump having a hole to form a gliding structure with the salient.

7. The retained needle structure as claimed in claim 1, wherein each of the fastening portions is replaced with a plate-like structure.

8. The retained needle structure as claimed in claim 1, wherein the hook portions of the syringe cover is an alternating hook combined with the stop portion of the catheter so as to fix the catheter.

9. The retained needle structure as claimed in claim 1, wherein each of the fastening portions of the syringe needle is replaced with a guide structure having a circular hole into which the respective gliding portion is inserted, each of the gliding portions has a stop portion at the rear end thereof and the respective stop portion has a salient formed thereon.

10. The retained needle structure as claimed in claim 1, wherein the fastening portions are replaced with a plurality of guide rails extending beside the syringe needle and each of the gliding portions at the rear end of the syringe cover has a bump having a hole at the rear end thereof, each of the plurality of guide rails has a protruding circular structure formed at a position near a front end of the guide rail and each of the holes runs through a corresponding one of the respective sliding rails.

11. The retained needle structure as claimed in claim 1, wherein the upper end of the syringe cover is combined with a stop structure through the connection member, wherein the stop structure is composed of a front stop body, a rear stop body and at least a curved piece, the curved piece being connected between the front stop body and the rear stop body, the curved piece has a curved portion at a central portion thereof and the rear end of the read stop body is fastened with a fastening seat of the syringe cover.

12. The retained needle structure as claimed in claim 1, wherein the fastening portion of the syringe needle is replaced with an outer sleeve made of a thin and soft material and a sleeving portion connected thereto, wherein the sleeving portion is sleeved on the outer rim of the syringe needle and covered by a stop structure at an outer rim thereof, the stop structure being connected to the rear end of the syringe cover through a connection member at a front end thereof and urging the sleeving portion of the syringe needle at a rear end thereof.

13. The retained needle structure as claimed in claim 1, wherein the fastening portion of the syringe needle is replaced with an inner rim sleeve, at least an intermediate sleeve sleeved on the inner rim sleeve and an outer rim sleeve sleeved on the at least an intermediate sleeve, wherein the outer rim sleeve is sleeved on the outer rim of the syringe needle at a rear end thereof, each of the inner rim sleeve and the intermediate sleeve has a ring-shaped protrusion at an outer rim of the rear end thereof and the inner rim sleeve has a plurality of hook portions at an inner rim of a front end thereof.

14. The retained needle structure as claimed in claim 1, wherein the fastening portion of the syringe needle is replaced with a plurality of guide slots formed within the syringe needle, wherein a respective one of the gliding portions of the syringe cover passes through each of the plurality of guide slots, either or all of the gliding portions has a stop body at the rear end thereof.

15. The retained needle structure as claimed in claim 1, wherein the fastening portions of the syringe needle is replaced with a plurality of sliding slots formed within the syringe needle, wherein a respective one of the gliding portions of the syringe cover passed through each of the sliding slots and the syringe cover has a fastening slot at the upper end thereof, the respective gliding portions having a bump.

16. The retained needle structure as claimed in claim 1, wherein the syringe needle has a plurality of sliding rails at the outer rim thereof, wherein a respective one of the gliding portions at the rear end of the syringe cover passes through each of the plurality of sliding rails is inserted with and either or all of the gliding portions has a stop body at a rear end thereof.

17. The retained needle structure as claimed in claim 1, wherein the fastening portion of the syringe needle is replaced with a stop portion, a reception portion and fastening hooks, wherein the stop portion and the reception portion are formed at the front end of the outer rim of the syringe needle at rear end of the outer rim of the syringe needle, respectively, and the fastening hooks are disposed at the distal end of the syringe cover so as to hook one of the reception portion and the stop portion.

18. The retained needle structure as claimed in claim 17, wherein the syringe cover is a hollow structure and the syringe needle has an urging piece at an upper end thereof.

19. The retained needle structure as claimed in claim 1, wherein the cannula of the catheter has an inverse flow stopping structure formed by a plurality of blades along the inner rim of the cannula.

20. The retained needle structure as claimed in claim 1, wherein the cannula of the catheter has a stop body having a through-hole therein and is connected to a piston made of a resilient material through a spring at a front end thereof, wherein the piston has a plurality of guide slots thereon and is received within the through-hole of the stop body through an urging piece at a rear end thereof, the urging piece has a guide portion having an outer diameter smaller than the urging piece at a rear end thereof, the guide portion having an urging body extending at an outer rim thereof.

21. The retained needle structure as claimed in claim 1, wherein the fastening portion of the syringe needle is replaced with guide slots formed within the syringe needle and each corresponding to a respective of the plurality of gliding portions of the syringe cover in topology, wherein each of the guide slots has a stop body at a front end thereof, the stop body is fastened with a stop body at a rear end of the respective one of the plurality of gliding portions of the syringe cover so as to prevent the syringe needle and the syringe cover from separating with each other.

22. The retained needle structure as claimed in claim 1, wherein the syringe cover further has an urging piece with which the syringe needle pulled the injection needle along the sliding rails of the syringe cover out from an injected portion of a patient.

23. The retained needle structure as claimed in claim 1, wherein each of the sliding slots of the gliding portions at the rear end of the syringe cover has a plurality of fastening holes, wherein each of the fastening holes of the sliding slots has a protruding circular structure at a joint of the fastening holes and each the fastening potions of the syringe needle has a curved portion having a fastening piece fastened into within a respective one of the fastening holes at a top end thereof.

24. The retained needle structure as claimed in claim 1, wherein the catheter further comprises a fixation piece at an inner portion thereof.

25. The retained needle structure as claimed in claim 1, wherein the catheter has an adhesive piece being adhesive at two sides thereof for adhering the catheter to an injection portion of a patient.

26. The retained needle structure as claimed in claim 1, wherein the catheter has a fixation piece at an inner portion thereof, wherein the fixation piece has an urging body at a front end thereof, the urging body having a hollow tube and extending between the fixation piece and the stop body.

27. The retained needle structure as claimed in claim 1, wherein the catheter has a spring at an inner portion thereof, wherein the spring has an urging body formed at a front end thereof, the urging body having an irregular surface in topology and disposed between the spring and the stop body.

28. A retained needle structure, comprising:
a syringe needle having an injection needle at a front end thereof, an injection needle inserted on the insertion portion, at least a fastening portion at a side rim thereof and a plug for reflowing at a rear end thereof,
a catheter having a cannula, a flexible tube extending at a front end of the cannula and a stop portion disposed on an outer rim of a rear end of the cannula;
a syringe cover having a plurality of gliding portions extending at an outer rim of a rear end thereof, the plurality of gliding portions corresponding to the fastening portions of the syringe needle in position and amount, each of the gliding portions having a sliding slot formed thereon and a salient formed at a position near a distal end of each of the sliding slots,
wherein the syringe cover is sleeved on a front end of the syringe needle, the injection needle on the syringe needle runs through the syringe cover, the catheter is sleeved on the syringe cover through a cannula thereof, the fastening portions of the syringe needle is received within the respective sliding slots of the syringe cover so that the injection needle slides on the syringe needle through the sliding slots.

29. The retained needle structure as claimed in claim 28, wherein the respective sliding slot of each of the gliding portions has a downward slant portion formed at a position near the distal end thereof.

30. The retained needle structure as claimed in claim 29, wherein the downward slant portion has a reception slot disposed at a lower end thereof.

31. The retained needle structure as claimed in claim 28, wherein the injection needle has at least a fastening piece fastened with the stop portion of the catheter so that the catheter and the syringe cover have an enhanced fixation therebetween.

32. The retained needle structure as claimed in claim 28, wherein the injection needle of the syringe cover has one of at least a salient and at least an indentation formed thereon, which mates closely with at least an indentation and at least a salient of the urging portion of the catheter so that the catheter and the syringe cover have an enhanced fixation therebetween.

33. The retained needle structure as claimed in claim 32, wherein the at least an indentation runs through one of the injection needle and the urging portion.

34. The retained needle structure as claimed in claim 28, wherein the fastening portion of the syringe needle is replaced with a guide structure having a circular hole inserted by the gliding portions at the rear end of the syringe cover, wherein each of the gliding portions has a slant portion at a distal end thereof, the slant portion having a salient.

35. The retained needle structure as claimed in claim 28, wherein the syringe needle has a plurality of “n”-shaped guide rails extending at a side thereof and each of the gliding portions at the rear end of the syringe cover has a bump having a respective hole at the rear end thereof, wherein each of the “n”-shaped guide rails has a salient formed at a position near a front end thereof and the respective hole is inserted and thus combination with a respective one of the plurality of “n”-shaped guide rails.

36. The retained needle structure as claimed in claim 28, wherein the syringe cover further has an urging piece disposed at the front end of the syringe cover.

37. The retained needle structure as claimed in claim 28, wherein the fastening portions of the syringe needle is replaced with guide slots formed within the syringe needle, wherein each of the guide slots correspond to a respective one the plurality of gliding portions of the syringe cover in topology and has a stop body disposed at a front end thereof, the stop body is fastened with a stop body at the rear end of the respective gliding portion of the syringe cover.
